(19) United States
(12) Patent Application Publication
(10) Pub. No.: US 2024/0212844 A1
(43) Pub. Date: Jun. 27, 2024

(54) CHRONIC PAIN INTERNET PLUS MANAGEMENT PLATFORM AND CONSTRUCTION METHOD THEREOF

(71) Applicant: Affiliated Hospital of Nantong University, Nantong (CN)

(72) Inventors: Xiang ZHU, Nantong (CN); Chenlu JIANG, Nantong (CN); Jiacheng ZHAO, Nantong (CN); Yibin QIN, Nantong (CN); Xiaqing MA, Nantong (CN); Cuie LU, Nantong (CN); Qiang GU, Nantong (CN); Weiwei ZHOU, Nantong (CN); Can ZHOU, Nantong (CN); Jiapei CAI, Nantong (CN); Zhusheng CHEN, Nantong (CN); Ming LI, Nantong (CN)

(73) Assignee: Affiliated Hospital of Nantong University, Nantong (CN)

(21) Appl. No.: 18/288,017

(22) PCT Filed: Jun. 24, 2022
(86) PCT No.: PCT/CN2022/101017

§ 371 Date: Oct. 23, 2023

(30) Foreign Application Priority Data
Jun. 25, 2021 (CN) 202110716601.2

(51) Int. Cl. G16H 50/20; G16H 10/60; G16H 50/30

(52) U.S. Cl. CPC G16H 50/20; G16H 10/60; G16H 50/30

(57) ABSTRACT

A chronic pain Internet plus management platform is provided to perform file management for patients with chronic diseases by taking multi-dimension and multi-level information about patients as a starting point for chronic disease diagnosis, taking respective hospitals and home personal mobile devices as an information collecting media and integrating with an Internet chronic disease cloud database, thereby realizing AI-assisted screening, and intelligent analysis and prediction of health risks. Anew mode for chronic pain management is established by the new generation of information technology, such as mobile Internet, cloud computing and big data; meanwhile, self-management of the patient is assisted by the mode for chronic pain management to improve prognosis of chronic pain.

### CROSS REFERENCE TO RELATED APPLICATION

This patent application claims the benefit and priority of Chinese Patent Application No. 202110716601.2, entitled “Chronic Pain Internet Plus Management Platform and Construction Method Thereof” filed on Jun. 25, 2021, which is incorporated by reference herein in its entirety as part of the present application.

### TECHNICAL FIELD

The disclosure relates to the field of Internet applications, in particular to a chronic pain Internet plus (Internet+) management platform and a construction method thereof.

### BACKGROUND ART

The full name of chronic disease is noninfectious chronic disease (NCD), which is not a certain disease specifically, but is a summarization for some kind of disease with hidden onset, long and protracted course of disease, lack of definite evidence of infectious biological etiology, complex etiology, or complete disconfirmation. The chronic pain is currently one of the most harmful chronic diseases to people.

Chronic pain refers to pain that lasts for more than one month. Some people also metaphorically express chronic pain as an immortal cancer. Currently, there are at least 100 million patients with chronic pain in China. Chronic pain is one of the main problems of modem human health and social development, and is the second most common disease next only to upper respiratory infection in North America. Chronic pain is also one of the medical difficult problems concerned by clinical multidisciplines. The people suffering from chronic pain is as high as 30% of the total population in the developed countries around the world. For example, about 70 million people in the United States suffer from various levels of chronic pain, and chronic low back pain results in a loss of tens of billions of dollars per year.

In China, the main complaint about pain accounts for a considerable proportion of outpatients. Most of the patients suffering from pain have been diagnosed or treated by more than one specialist. Meanwhile, data shows that the chronic pain patients whose life quality is affected by pain have reached 20%-46% among the elderly living in communities in China. The morbidity of chronic diseases is as high as 73% among the elderly living in nursing homes, but less than 65% of these people with chronic pain visit medical institutions, and less than 28% of patients receive traditional medicine treatment.

### 1. Disadvantages in the Diagnosis and Treatment of Chronic Pain Limit the Improvement of its Treatment Effect.

Since chronic pain is a long-term and lasting disease, people often live with the “pain” and physical and mental suffering for a long time. In order to eliminate suffering, patients often seek help from multiple hospitals and departments, but the treatment effect of chronic pain is often unsatisfactory. Many patients gradually lose confidence and patience after many visiting to the doctor for many times. There are three main reasons as follows. First, chronic pain is also a clinical multidisciplinary problem, most of chronic pain needs to be diagnosed jointly by multiple departments and the resources available to patients at one outpatient clinic are very limited, which greatly limits the treatment of chronic pain. Secondly, it is related to the limited medical resources in China, which results in difficulty of getting better treatment and management of chronic pain for many primary communities. Thirdly, many chronic pain patients need medical treatment many times, but the disease cannot be improved quickly, and the current primary medical security system of China is not perfect, which undoubtedly causes great damage to economy of patients.

The causes of chronic diseases, in particular chronic pain, are extremely complex, so that it is difficult for a single department to accurately diagnose and treat them. In China, the main complaint about pain accounts for a considerable proportion of outpatients. Most of the patients suffering from pain have been diagnosed or treated by more than one specialist. However, based on the current knowledge scope about anatomy, physiology, pathology and other related subjects that we have mastered, it is difficult to determine a diseased site and involved innervation mechanism for each patient, and to make different diagnoses and choose a reasonable treatment method, thus resulting in many chronic patients being transferred between various departments of various hospitals, which causes economic losses and mental harm to the patients. At present, a disadvantage for chronic disease treatment is that it is difficult to accurately determine and treat chronic diseases caused by multiple factors as the outpatient clinics of central hospitals in China are mostly diagnosed by specialists, while general practitioners in primary hospitals are limited by equipment and technology such that they cannot give effective diagnosis to patients, and multi-department consultation is generally used for intractable diseases with higher harmfulness, and thus there are few consultation opportunities for chronic diseases. Moreover, due to lack of effective interconnection and management of information, effective and ineffective means of diagnosis and treatment cannot be summarized for learning, which greatly limits the improvement of chronic disease treatment technology.

### 2. Internet Brings a New Opportunity for Chronic Disease Management.

With the advent of the Internet era, new-generation information technologies, such as big data, mobile Internet, Internet of Things and cloud computing, will drive the rapid growth of Internet medicine. Wearable devices may intelligently and automatically collect data of various physiological indicators of a human body anytime and anywhere and the data may be transmitted timely to the cloud for storage and processing, which changes the traditional consultation for doctors of “watching, touching, tapping and listening”. The big data technology enables the medical and health industry to enter anew era of “accurate and personalized diagnosis and treatment”. The cloud computing is promoting the transformation of the medical mode from disease medicine to health medicine, and from focus on treatment to prevention and treatment. The extensive application of mobile Internet breaks the original boundaries of the field of medicine and health. The development of these new technologies brings a new opportunity for chronic disease management.

Properties of Internet+medicine may perfectly solve the current difficulties existing in the diagnosis and treatment of chronic diseases. First, Internet big data may summarize the features, diagnosis and treatment technologies, and medicine recommendations for various chronic diseases in the current primary and central hospitals as well as top Grade 3A hospitals in China, which may help primary doctors to make accurate diagnosis. Secondly, Internet+technology supports online services such as mobile phones and websites, and patients may consult online, which greatly facilitates patients with chronic diseases. Patients do not need to register in multiple departments of multiple hospitals for diagnosis and treatment, which not only reduces overload of the hospital system, but also reduces the economic losses and mental harm to the patients. Finally, Internet medicine may summarize and count fragmentary information about diagnosis of patients of primary hospitals. Database may be continuously completed by cloud computing to improve the diagnosis and treatment technology of chronic diseases.

Chronic pain has a long formation cycle, high specialization and complexity. At present, the fragmented medical and health service system in China and the provider-centered service mode are far from meeting the needs of chronic pain prevention and control. The CPC Central Committee and State Council has issued the “Outline of the Healthy China 2030 Plan”, indicating “focus on rural and primary areas”, “accelerate the transformation of the development mode in the health field, and maintain and guarantee people's health in an all-round and full-cycle way” and “transform the service mode and build an integrated medical and health service system”, which has pointed out the direction for the development of health field in China.

China actively promotes chronic prevention and control, but still faces some problems, for example, primary medical institutions have not become the main force of chronic disease management, people have poor knowledge of chronic diseases, difficult self-health management and poor compliance, and it is difficult for doctors diagnose and treat accurately. With the advent of the Internet era, the development of new technologies has brought a new opportunity for chronic disease management.

### 3. The Role of Internet Medicine in the Diagnosis and Treatment of Chronic Diseases in a Special Period:

During the prevention and control of the COVID-19 pandemic, how to reduce the risk of cross-infection as well as to ensure timely treatment, further consultation and safe medication for patients with chronic diseases is a major problem faced by the government, hospitals and patients, and the Internet hospitals are the direction to solve the problem, which reassure patients with chronic diseases. The first batch of Internet hospitals admitted in Hunan has been officially opened. Wenzhou, Ningbo and other places in Zhejiang also provide convenience services for patients, especially those with chronic diseases, such as further consultation, prescription and medicine delivery, through the relevant platform of “Internet+medicine”. Ningbo “Cloud Hospital” platform is responsible for reviewing online prescriptions issued by primary medical institutions according to the Internet diagnosis and treatment standards. The settled pharmaceutical trading enterprises provide medicine dispensing, logistics distribution and other services, and the pharmaceutical trading enterprises may entrust a third party for distribution.

In addition to government-led Internet hospitals with stronger local attributes, Internet hospitals invented by society and enterprises are also taking active action. For example, a pharmaceutical e-commerce giant Alibaba Health Information Technology Limited announced that it would launch a “chronic disease welfare plan” with nearly 50 pharmaceutical enterprises, covering multi-dimension service range such as prescription for further consultation, medicine distribution, and chronic disease management. It is particularly important to establish effective communication among Internet hospitals, the society and patients. These hospitals have doctors with online diagnosis and treatment qualifications. The doctor will determine whether the user's disease is stable and suitable for using these medicines according to the medicine information and relevant follow-up information submitted by the user, so as to decide whether to prescribe. After that, it will go to the process of medicine purchase. There will be a dedicated prescription review team of pharmacists to finally review the prescription information. After the review is passed, it will enter a pharmacy process. The pharmacy will check the prescription and order information, and then deliver the medicines.

As more and more attention has been paid to the difficulty of dispensing and obtaining medicines for chronic diseases during the epidemic, the National Health Commission has released a clear signal to encourage online further consultation of some chronic diseases and medicine delivery services. Meanwhile, the National Healthcare Security Administration has also made it clear that a “long prescription” reimbursement policy will be implemented, which relaxes the prescription amount of patients with chronic diseases, such as hypertension and diabetes, to three months for ensuring a long-term medication demand of insured patients. Beijing and Shanghai have taken action.

Fang Laiying, vice president of Chinese Hospital Association, said in an interview that for the supply of medication of chronic diseases, some new situations and experiences during the epidemic are worthy of further policy research and improvement.

Chronic pain is a complex clinical problem. It is difficult for a single discipline to quickly complete the diagnosis and treatment, and thus patients often suffer from long-term pain. Primary community medical service institutions are the main performers of chronic pain management. However, due to the excessive concentration of high-quality medical resources to large medical institutions, primary medical institutions lack general practitioners and preventive health care doctors who may undertake chronic pain management, and also lack adequate service facilities and medical equipment. This has resulted in insufficient treatment of chronic pain at the primary medical institution. On the other hand, in recent years, although medical institutions have increased their efforts to screen related chronic pain, they all work independently with fragmented information and management. In particular, the health examination center of the primary hospital only stays in the telephone follow-up and oral simple education for the high-risk groups of chronic diseases, and lacks continuity in providing services for people with chronic pain. The general practice department of the hospital goes to the community to carry out activities in a widely publicized manner, which lacks pertinence.

### SUMMARY

The technical problem to be solved by the present disclosure is to provide a chronic pain Internet plus management platform and a construction method thereof. A new mode for chronic pain management is established by the new generation of information technology, such as mobile Internet, cloud computing and big data to open up a new way for chronic pain diagnosis and personalized treatment.

In order to solve the above technical problems, the embodiments of the present disclosure provide a chronic pain Internet plus management platform configured to perform file management for patients with chronic diseases by taking multi-dimension and multi-level information about patients as a starting point for chronic disease diagnosis, taking respective hospitals and home personal mobile devices as an information collecting media and integrating with an Internet chronic disease cloud database, thereby realizing AI-assisted screening, and intelligent analysis and prediction of health risks.

In an embodiment, the chronic pain Internet plus management platform includes a chronic disease database, a region chronic disease support platform, a hospital chronic disease business subsystem, a community hospital chronic disease business subsystem, an offline chronic disease service center, and an online home health service system, wherein
the chronic disease database exchanges data with the region chronic disease support platform mutually;
the hospital chronic disease business subsystem, the community hospital chronic disease business subsystem and the online home health service system exchange data respectively with the region chronic disease support platform mutually;
the hospital chronic disease business subsystem is controlled and managed by a region central hospital;
the community hospital chronic disease business subsystem is controlled and managed by a community health service center; and
the offline chronic disease service center and the online home health service system provide service for home patients.

In an embodiment, the region chronic disease support platform includes a telemedicine module, a remote electrocardiogram module, a remote image module, a decision and analysis module, an artificial intelligence AI engine, a dual referral module and a data integration platform.

In an embodiment, the hospital chronic disease business subsystem includes an online prescription module, an online diagnosis and treatment module, a treatment plan formulation module and a specialist follow-up module.

In an embodiment, the community hospital chronic disease business subsystem includes an electronic filing module, a treatment plan execution module, an outpatient counterparty module and a chronic disease management module.

In an embodiment, the offline chronic disease service center includes a wearable device experience center and a contract consulting desk, and the online home health service system comprises a personal file module, a member incentive module, an online mall, a video consultation module, a data monitoring module and a health education module.

The present disclosure also provides a construction method of the chronic pain Internet plus management platform, including following steps:
S1: establishing a chronic disease database, which includes collecting information of primary patients, coordinating data of respective primary hospitals, and establishing a data platform and a computing power platform by a big data center and a computing center to provide data services for a data bank and provide computing power support for an algorithm model;
S2: building a region chronic disease support platform, wherein the region chronic disease support platform comprises a telemedicine module, a remote electrocardiogram module, a remote image module, a decision and analysis module, an artificial intelligence AI engine, a dual referral module and a data integration platform;
S3: establishing a hospital chronic disease business subsystem in a region central hospital, integrating resources of medical units in a region to provide a new management mode for the medical units, and establishing a chronic pain management plan comprising auxiliary diagnosis, treatment, and follow-up to realize standardization of pain specialist multi-medical center data and space vector transformation of pain specialist clinical phenotype data;
S4: establishing a community hospital chronic disease business subsystem in a community health service center, collecting fragmentary information of the primary patients to form a chronic pain diagnosis case bank for reference by general practitioners in the community which lacks technology and equipment;
S5: establishing an offline chronic disease service center to provide chronic disease patients with wearable devices for experiencing; and
S6: establishing an online home health service system to collect information of the chronic disease patients for summarizing and recording, and provide services about online appointments, online consultations and auxiliary health management, wherein the information of the chronic disease patients comprise personal information, personal medical cases and treatment history.

The present disclosure also provides an operating method of the Internet plus management platform for chronic pain, including following steps:
S101: establishing resident health files, and intelligently screening chronic diseases of target population, including
establishing an Internet of Things collection system, based on the resident health files and a screening standard for chronic pain high-risk population, to improve resident health information and provide data support for hierarchical diagnosis and treatment; collecting questionnaire data corresponding to high-risk conditions and a set of sample data about whether a sample is diagnosed as the chronic pain in a sample population, and combining the questionnaire data and the set of sample data with the resident health files to form the sample data (with the resident health files, high-risk condition questionnaires, and whether the sample is diagnosed as the chronic pain);
S102: sharing information between an Internet analysis and diagnosis platform and a region medical information system, including
establishing an Internet hierarchical diagnosis and treatment platform and interconnecting the resident health files and electronic cases with the Internet hierarchical diagnosis and treatment platform to realize medical information sharing and medical service collaboration in a region; and interconnecting the hierarchical diagnosis and treatment platform with existing region health information platforms, all-level medical institutions and region medical central business systems to assist doctors to easily access to anamnesis and specific conditions about diagnosis and treatment of patients through platforms, and provide data support for implementation of the hierarchical diagnosis and treatment; and
establishing a standard knowledge system and service process and an uniform diagnosis and treatment standard and operating specification to improve service quality of primary medical institutions; transforming an original one-to-one service mode for large medical institutions and primary medical institutions to a many-to-many service mode by support of a new generation of information technology, so as to enable the large medical institutions to focus on solving intractable diseases and critical illnesses, enable the primary medical institutions to emphatically focus on common diseases, chronic diseases, frequently-occurring diseases and rehabilitation services, to support construction of a hierarchical diagnosis and treatment mode for “separate treatment of acute and chronic diseases, primary diagnosis, dual referral and superior-subordinate collaboration”;
S103: assisting diagnosis and treatment of chronic pain by interoperability of platforms, including
assisting patients to be diagnosed by platform, including: based on construction of a patient information platform and an Internet triage platform, realizing collaboration medical services for intelligent triage, appointment for registration, appointment for diagnosis and treatment, remote outpatient service, telemedicine, dual referral/inspection, remote instruction, and communication between doctors and patients, and between doctors and doctors, etc.; and assisting doctors in task management, task recommendation and automatic distribution, by an informatization platform and a method for intelligent recommendation and standardized process design, to improve standardized diagnosis and treatment capabilities of primary doctors for the chronic pain; and
assisting primary doctors in precise diagnosis and treatment, including: based on an assessment for conditions of a patient, assessing a disease group that the patient belongs to, comprehensively assessing a disease level and a treatment plan by combining with information of a platform database, and formulating a medication recommendation based on recommendations given by the platform and a hospital situation, and submitting data; establishing files of respective primary patients, comprising information about a health file, an electronic case, clinical features, the disease group, the medication recommendation, a condition whether the disease is acutely exacerbated and a condition whether there is a complication, to form a systematic diagnosis and treatment report, and building an acute exacerbation risk model and a complication occurrence risk model based on patient information and different medication plans to formulate a medication plan; modeling abnormal data, based on data of the resident health files, medication situation and medication effect for patients accumulated in each region, in combination with abnormal data beyond medication specification, and building a hybrid model supporting grouping of intractable diseases and an abnormal modeling model, to precisely recommend medication; and
S104: performing intelligent medication guidance and auxiliary follow-up for chronic pain, including
formulating an optimum medication plan by cloud computing, based on follow-up information comprising the resident health files, the clinical features, the medication situation and a condition whether the disease is acutely exacerbated, in combination with information of the database to give patients medication guidance at any time, and performing regular auxiliary follow-up according to cycles of different chronic pain diseases, and recording and uploading patient information to the cloud to formulate an appropriate recommendation for follow-up plan.

The above technical scheme of the present disclosure has following beneficial effects:
1. In the present disclosure, a new mode for chronic pain management is established by the new generation of information technology, such as mobile Internet, cloud computing and big data; meanwhile, self-management of the patient is assisted by the new mode for chronic pain management to improve prognosis of chronic pain.
2. The “Internet+” chronic pain management mode of the present disclosure opens up a new way for chronic pain diagnosis and personalized treatment, so that the management of chronic pain is no longer limited to a traditional management mode, which is an advancement of medical and health care with the times, and also a development tendency of chronic pain management. Patients may deeply understand the development of their own chronic diseases, by the “Internet+” chronic pain management mode, to improve their self-management ability. Meanwhile, medical institutions may directly obtain examination results and medication situation of patients by Internet, which is conducive to promote real-time communication between medical staff and patients, and promote communication between patients.

### BRIEF DESCRIPTION OF THE DRAWINGS

In order to illustrate the embodiments of the present disclosure or the technical solutions of the conventional art more clearly, the accompanying drawings used in the embodiments will be briefly described below. Obviously, the accompanying drawings described below show merely some embodiments of the present disclosure. For those skilled in the art, other drawings can be obtained according to the accompanying drawings without creative efforts.

FIG. 1 is a block diagram of the present disclosure;

FIG. 2 is a data hierarchical structure diagram of the present disclosure.

### DETAILED DESCRIPTION OF THE EMBODIMENTS

The technical solutions in the embodiments of the present disclosure will be described clearly and completely below in conjunction with the accompanying drawings in the embodiments of the present disclosure. Obviously, the described embodiments are merely a part of the embodiments of the present disclosure, rather than all of embodiments. All other embodiments obtained by ordinary technicians in the art based on the embodiments of the present disclosure without creative efforts shall fall within the scope of the present disclosure.

To make the above purposes, features and advantages of the present disclosure clearer and more comprehensible, the present disclosure is described in further detail below in conjunction with the accompanying drawings and specific implementations.

“Internet+”, as an efficient and fast medium for chronic pain management, utilizes information technologies to realize intelligent monitoring and guiding education of chronic pain, and provide convenience for remote collaboration of medical staff and optimized utilization of medical resources, which may be widely used in the management of patients with chronic pain. Therefore, medical staff should follow the trend to actively participate in medical informatization course training for improving their own information technology level. The medical staff should focus on the attitude and experience of patients for the new form of services, participate in the improvement of the application of “Internet+” technology, and provide a social support platform in a new form for patients with chronic pain by using network tools. In addition, hospitals are also suitable for applying the “Internet+” educational technology to create convenient learning conditions for medical staff, and carrying out research on improving the management efficiency of “Internet+” technology by using quality management tools. Meanwhile, since health management is implemented by the “Internet+” technology, private information such as patient health data has a risk of leakage. And since the Internet carries a large amount of information, misleading information channels may pose a threat to health of patients. Therefore, it requires government departments to participate in supervision and formulate “Internet+” technical health management regulations, and requires Internet engineers to assist for ensuring information security, thereby improving the standardization of “Internet+” health management, so that hospitals and communities may provide patients with correct and effective health services to further enhance the role of “Internet+” technology in the chronic pain management.

In order to fully apply “Internet+” technology to the chronic pain management, the collaboration of the following departments is required:

### 1) Government Departments

Relevant government departments have authority and credibility, and are responsible for the health management of residents. Therefore, relevant government departments need to give full play to their advantages to formulate relevant policies, rules and regulations of the “Internet+” chronic disease management mode. And, the health administrative department also needs to strengthen the supervision of the medical platform to make the use of the medical platform more standardized. In addition, medical and related institutions also need to fully gather forces in various aspects to conduct publicity and education on “Internet+” chronic disease management for the public, so that the “Internet+” chronic disease industry may achieve healthy and sustainable development.

### 2) Chronic Disease Medical Institutions

Chronic disease medical institutions (including primary medical and health institutions, rehabilitation hospitals, and nursing homes), as executors of the “Internet+” chronic disease management mode, should try their best to open up and share resources, and jointly serve for the condition monitoring, medication guidance and follow-up question answering of chronic diseases in their respective regions.

### 3) Patients

In order to better manage their chronic pain, patients need to actively learn the chronic pain network operating system, actively monitor their own physiological indicators, actively communicate with medical staff, and enjoy the convenience brought by the new management mode.

### 4) Software Developers

Developers of mobile medical platforms should take needs of the public as a design objective to make the software more humanized, thereby providing patients with high-quality services. When developing and designing applications, it is necessary to fully consider the acceptance level of the public, and appropriately simplify the application process of chronic pain management. Meanwhile, it is necessary to use traditional means such as brochures, radio and television to publicize, so that the public gradually accept and ultimately benefit from the “Internet+” chronic pain management mode. In addition, developers of the platform should cooperate with medical institutions to obtain better medical resources and promote the development of mobile medical platform software.

### 5) Insurance Company

At present, the role of insurance companies in the “Internet+” chronic disease management is still very limited. On one hand, medical insurance only compensates for expenses incurred by diseases, not for the expenses incurred by rehabilitation care, chronic diseases and health management. On the other hand, the development of the “Internet+” chronic disease management mode will definitely be limited without medical insurance. Therefore, insurance companies should firmly grasp an opportunity to expand the medical insurance market.

The “Internet+” chronic disease management mode will be a new type of management mode. For this purpose, the present disclosure provides the Internet+management platform for chronic pain, which is configured to perform file management for patients with chronic diseases by taking multi-dimension and multi-level information about patients as a starting point for chronic disease diagnosis, taking respective hospitals and home personal mobile devices as an information collecting media and integrating with an Internet chronic disease cloud database, thereby realizing AI-assisted screening, and intelligent analysis and prediction of health risks.

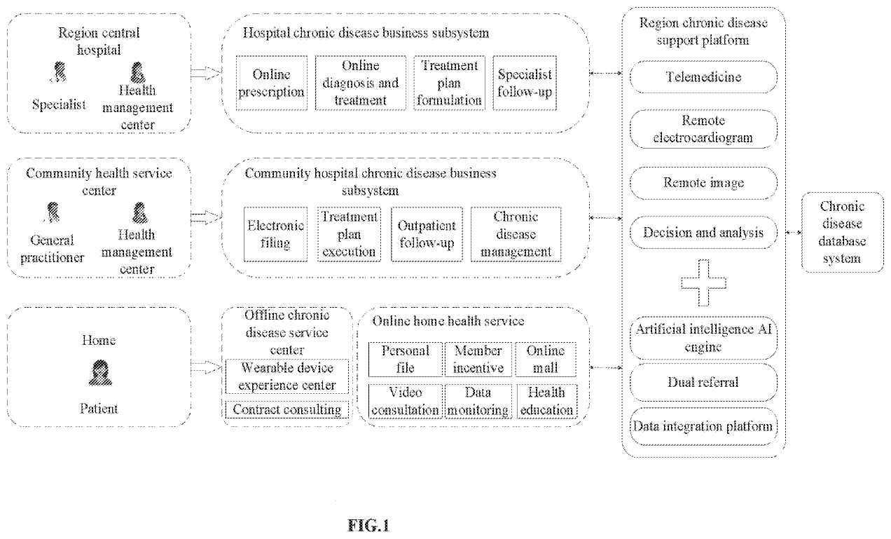

As shown in FIG. 1, the chronic pain Internet+management platform includes a chronic disease database, a region chronic disease support platform, a hospital chronic disease business subsystem, a community hospital chronic disease business subsystem, an offline chronic disease service center, and an online home health service system.

The chronic disease database exchanges data with the region chronic disease support platform mutually.

The hospital chronic disease business subsystem, the community hospital chronic disease business subsystem and the online home health service system exchange data respectively with the region chronic disease support platform mutually.

The hospital chronic disease business subsystem is controlled and managed by a region central hospital.

The community hospital chronic disease business subsystem is controlled and managed by a community health service center.

The offline chronic disease service center and the online home health service system provide service for home patients.

Specifically, the region chronic disease support platform includes a telemedicine module, a remote electrocardiogram module, a remote image module, a decision and analysis module, an artificial intelligence AI engine, a dual referral module and a data integration platform.

The hospital chronic disease business subsystem includes an online prescription module, an online diagnosis and treatment module, a treatment plan formulation module and a specialist follow-up module.

The community hospital chronic disease business subsystem includes an electronic filing module, a treatment plan execution module, an outpatient counterparty module and a chronic disease management module.

The offline chronic disease service center includes a wearable device experience center and a contract consulting desk, and the online home health service system includes a personal file module, a member incentive module, an online mall, a video consultation module, a data monitoring module and a health education module.

The present disclosure also provides a construction method of the Internet+ management platform for chronic pain, including steps S1-S6.

In step S1, a chronic disease database is established. Specifically, information of primary patients is collected, data of respective primary hospitals is coordinated, and a data platform and a computing power platform are established by a big data center and a computing center to provide data services for a data bank and provide computing power support for an algorithm model.

In step S2, a region chronic disease support platform is built. The region chronic disease support platform includes a telemedicine module, a remote electrocardiogram module, a remote image module, a decision and analysis module, an artificial intelligence AI engine, a dual referral module and a data integration platform.

In step S3, a hospital chronic disease business subsystem is established in a region central hospital, resources of medical units in a region are integrated to provide a new management mode for the medical units, and a chronic pain management plan, including auxiliary diagnosis, treatment, and follow-up, is established to realize standardization of pain specialist multi-medical center data and space vector transformation of pain specialist clinical phenotype data.

In step S4, a community hospital chronic disease business subsystem is established in a community health service center, fragmentary information of the primary patients is collected to form a chronic pain diagnosis case bank for reference by general practitioners in the community who lack technology and equipment.

In step S5, an offline chronic disease service center is established to provide chronic disease patients with wearable devices for experiencing.

In step S6, an online home health service system is established to collect information of the chronic disease patients for summarizing and recording, and provide services about online appointments, online consultations and auxiliary health management, where the information of the chronic disease patients includes personal information, personal medical cases and treatment history.

In other words, the implementation of the present disclosure is as follows:

1) A basic platform is established at the initial term. Specifically, the information of primary patients is collected, and the data of respective primary hospitals is coordinated; and then the data platform and the computing power platform are established by the big data center and the computing center to provide data services for the data bank and provide computing power support for the algorithm model.

2) Data modeling at the medium term achieves uniform management of data, and standard Schema management achieves the standardization supporting modeling and data management. A knowledge map, as a highly explanatory decision model, provides services for the business layer. An algorithm model module is responsible for building various standardized algorithm models, and may provide various machine learning models such as classification, regression and optimization.

3) Finally, various business product modules are designed, and various AI applications are built in such a way of data+model.

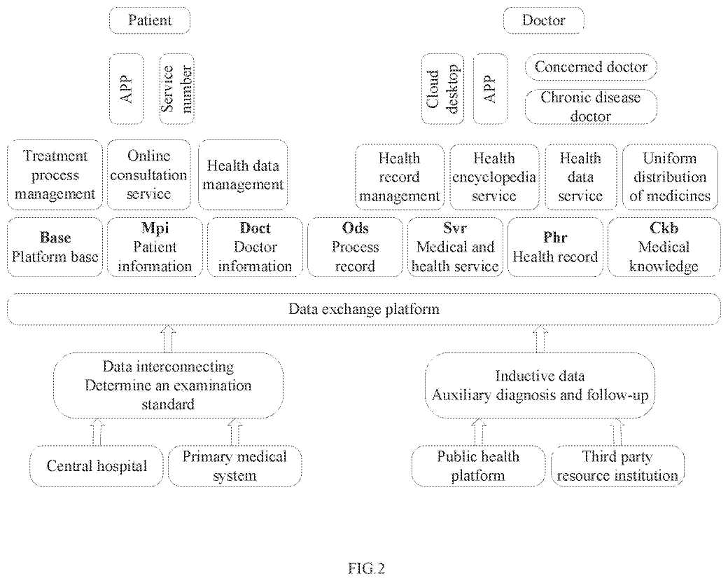

In order to build the Internet+management platform for chronic pain, the research content of the present disclosure is mainly divided into several levels, including patient level, doctor level, and hospital and related third party level. In the patient level, the personal information, personal medical records, treatment history and other information of patients are collected and recorded, and services such as online appointments, online consultations, and auxiliary health management are provided. In the doctor level, more cases about chronic pain diagnosis are provided for general practitioners who lack basic technology and equipment for their learn and reference, and more fragmentary information of the primary patients may be collected by doctors. In the hospital and related third party level, resources of related medical units in the region are integrated to provide the new management mode for the medical units, and a chronic pain management plan including auxiliary diagnosis, treatment, and follow-up is established. The research framework is shown as FIG. 2. The work that needs to be carried out includes:
1) informatization management of hospital data;
2) standardization of pain specialist multi-medical center data;
3) space vector transformation of pain specialist clinical phenotype data;
4) personalized diagnosis and treatment formulated according to the full phenotype clinical data matching database of patients; and
5) establishment of chronic pain patient files to provide self-management strategies, chronic pain management follow-up support, etc.

Based on the above, the present disclosure also provides an operating method of the Internet+management platform for chronic pain, including steps S101-S104.

In step S101, resident health files are established, and chronic diseases of target population are intelligently screened.

Specifically, based on the resident health files and a screening standard for chronic pain high-risk population, an Internet of Things collection system is established to improve resident health information and provide data support for hierarchical diagnosis and treatment. Questionnaire data corresponding to high-risk conditions and a set of sample data about whether a sample is diagnosed as the chronic pain in a sample population are collected. And the questionnaire data and the set of sample data are combined with the resident health files to form the sample data.

In step S102, information is shared between an Internet analysis and diagnosis platform and a region medical information system.

Specifically, an Internet hierarchical diagnosis and treatment platform is established, and the resident health files and electronic cases are interconnected with the Internet hierarchical diagnosis and treatment platform to further realize medical information sharing and medical service collaboration in the region. The hierarchical diagnosis and treatment platform is interconnected with existing region health information platforms, all-level medical institutions and region medical central business systems to assist doctors to easily access to anamnesis and specific conditions about diagnosis and treatment of patients through the platforms, and provide data support for implementation of the hierarchical diagnosis and treatment.

A standard knowledge system and service process and an uniform diagnosis and treatment standard and operating specification are established to improve service quality of primary medical institutions. An original one-to-one service mode for large medical institutions and primary medical institutions is transformed to a many-to-many service mode by support of a new generation of information technology, so as to enable the large medical institutions to focus on solving intractable diseases and critical illnesses, enable the primary medical institutions to emphatically focus on common diseases, chronic diseases, frequently-occurring diseases and rehabilitation services, to support construction of a hierarchical diagnosis and treatment mode for separate treatment of acute and chronic diseases, primary diagnosis, dual referral and superior-subordinate collaboration.

In step S103, diagnosis and treatment of chronic pain are assisted by interoperability of platforms.

Specifically, patients are assisted to be diagnosed by platform. Based on construction of a patient information platform and an Internet triage platform, collaboration medical services are realized for intelligent triage, appointment for registration, appointment for diagnosis and treatment, remote outpatient service, telemedicine, dual referral/inspection, remote instruction, communication between doctors and patients, and between doctors and doctors, etc. And doctors are assisted in task management, task recommendation, automatic distribution, etc, by an informatization platform and a method for intelligent recommendation, standardized process design, etc., to improve standardized diagnosis and treatment capabilities of primary doctors for the chronic pain.

Primary doctors are assisted in precise diagnosis and treatment. A disease group that the patient belongs to is assessed based on an assessment for conditions of a patient. A disease level and a treatment plan are comprehensively assessed by combining with information of a platform database. And a medication recommendation is formulated based on recommendations given by the platform and a hospital situation, and data is submitted. The files of respective primary patients are established to form a systematic diagnosis and treatment report, where the files of the respective primary patients include information about the health file, the electronic case, clinical features, the disease group, the medication recommendation, the condition whether the disease is acutely exacerbated and the condition whether there is a complication. An acute exacerbation risk model and a complication occurrence risk model are built based on patient information and different medication plans to formulate a medication plan. Abnormal data is modeled based on data of the resident health files, medication situation and medication effect for patients accumulated in each region, in combination with abnormal data beyond medication specification. And a hybrid model supporting grouping of intractable diseases and an abnormal modeling model is built to precisely recommend medication.

In step S104, the intelligent medication guidance and auxiliary follow-up for chronic pain are performed.

Specifically, based on follow-up information including the resident health files, the clinical features, the medication situation and the condition whether the disease is acutely exacerbated, an optimum medication plan is formulated by cloud computing in combination with information of the database to give patients medication guidance at any time. And regular auxiliary follow-up is performed according to cycles of different chronic pain diseases, and patient information is recorded and uploaded to the cloud to formulate an appropriate recommendation for follow-up plan.

Since the use and promotion of “Internet+” will realize the characteristics of medical process optimization, efficiency maximization, quality optimization, evaluation automation, and scientific decision. Therefore, it is necessary to make full use of Internet mobile medical apps, cloud computing, big data and other technological means to facilitate the rapid development of the “Internet+” chronic disease management mode, which will further improve the quality of chronic pain management and reduce medical costs. The development direction of the future medical mode is that: medical staff can not only provide services for patients in medical institutions, but also monitor patients' conditions, guide medication and provide consulting services by Internet.

The present disclosure has following beneficial effects:

1: The present disclosure completes the construction of an Internet health management platform for chronic pain to provide regional medical services and reduce social medical expenses.

2: The present disclosure provides an efficient and precise prevention and management method for the prevention and control of chronic pain at the primary medical institutions.

In this specification, various embodiments are described in a progressive manner. Each embodiment focuses on the differences from other embodiments, and the same and similar parts among the various embodiments may refer to each other.

Specific examples are used herein for illustration of the principles and implementations of the present disclosure. The descriptions of the foregoing embodiments are used to help understand the method and core ideas of the present disclosure. At the same time, for the ordinary technicians in the art, there will be changes in the specific implementations and application scope according to the ideas of the present disclosure. In summary, the content of this specification should not be interpreted as a limitation of the present disclosure.

1. A chronic pain Internet plus management platform, wherein the chronic pain Internet plus management platform is configured to perform file management for patients with chronic diseases by taking multi-dimension and multi-level information about patients as a starting point for chronic disease diagnosis, taking respective hospitals and home personal mobile devices as an information collecting media and integrating with an Internet chronic disease cloud database, thereby realizing AI-assisted screening, and intelligent analysis and prediction of health risks.

2. The chronic pain Internet plus management platform according to claim 1, comprising a chronic disease database, a region chronic disease support platform, a hospital chronic disease business subsystem, a community hospital chronic disease business subsystem, an offline chronic disease service center, and an online home health service system, wherein
the chronic disease database exchanges data with the region chronic disease support platform mutually;
the hospital chronic disease business subsystem, the community hospital chronic disease business subsystem and the online home health service system exchange data respectively with the region chronic disease support platform mutually;
the hospital chronic disease business subsystem is controlled and managed by a region central hospital;
the community hospital chronic disease business subsystem is controlled and managed by a community health service center; and
the offline chronic disease service center and the online home health service system provide service for home patients.

3. The chronic pain Internet plus management platform according to claim 2, wherein the region chronic disease support platform comprises a telemedicine module, a remote electrocardiogram module, a remote image module, a decision and analysis module, an artificial intelligence AI engine, a dual referral module and a data integration platform.

4. The chronic pain Internet plus management platform according to claim 2, wherein the hospital chronic disease business subsystem comprises an online prescription module, an online diagnosis and treatment module, a treatment plan formulation module and a specialist follow-up module.

5. The chronic pain Internet plus management platform according to claim 2, wherein the community hospital chronic disease business subsystem comprises an electronic filing module, a treatment plan execution module, an outpatient counterparty module and a chronic disease management module.

6. The chronic pain Internet plus management platform according to claim 2, wherein the offline chronic disease service center comprises a wearable device experience center and a contract consulting desk, and the online home health service system comprises a personal file module, a member incentive module, an online mall, a video consultation module, a data monitoring module and a health education module.

7. A construction method of the chronic pain Internet plus management platform according to claim 1, comprising:
S1: establishing a chronic disease database, which comprises collecting information of primary patients, coordinating data of respective primary hospitals, and establishing a data platform and a computing power platform by a big data center and a computing center to provide data services for a data bank and provide computing power support for an algorithm model;
S2: building a region chronic disease support platform, wherein the region chronic disease support platform comprises a telemedicine module, a remote electrocardiogram module, a remote image module, a decision and analysis module, an artificial intelligence AI engine, a dual referral module and a data integration platform;
S3: establishing a hospital chronic disease business subsystem in a region central hospital, integrating resources of medical units in a region to provide a new management mode for the medical units, and establishing a chronic pain management plan comprising auxiliary diagnosis, treatment, and follow-up to realize standardization of pain specialist multi-medical center data and space vector transformation of pain specialist clinical phenotype data;
S4: establishing a community hospital chronic disease business subsystem in a community health service center, collecting fragmentary information of the primary patients to form a chronic pain diagnosis case bank for reference by general practitioners in the community which lacks technology and equipment;
S5: establishing an offline chronic disease service center to provide chronic disease patients with wearable devices for experiencing; and
S6: establishing an online home health service system to collect information of the chronic disease patients for summarizing and recording, and provide services about online appointments, online consultations and auxiliary health management, wherein the information of the chronic disease patients comprise personal information, personal medical cases and treatment history.

8. The construction method of the chronic pain Internet plus management platform according to claim 7, further comprising: a chronic disease database, a region chronic disease support platform, a hospital chronic disease business subsystem, a community hospital chronic disease business subsystem, an offline chronic disease service center, and an online home health service system, wherein
the chronic disease database exchanges data with the region chronic disease support platform mutually;
the hospital chronic disease business subsystem, the community hospital chronic disease business subsystem and the online home health service system exchange data respectively with the region chronic disease support platform mutually;
the hospital chronic disease business subsystem is controlled and managed by a region central hospital;
the community hospital chronic disease business subsystem is controlled and managed by a community health service center; and
the offline chronic disease service center and the online home health service system provide service for home patients.

9. The construction method of the chronic pain Internet plus management platform according to claim 8, wherein the region chronic disease support platform comprises a telemedicine module, a remote electrocardiogram module, a remote image module, a decision and analysis module, an artificial intelligence AI engine, a dual referral module and a data integration platform.

10. The construction method of the chronic pain Internet plus management platform according to claim 8, wherein the hospital chronic disease business subsystem comprises an online prescription module, an online diagnosis and treatment module, a treatment plan formulation module and a specialist follow-up module.

11. The construction method of the chronic pain Internet plus management platform according to claim 8, wherein the community hospital chronic disease business subsystem comprises an electronic filing module, a treatment plan execution module, an outpatient counterparty module and a chronic disease management module.

12. The construction method of the chronic pain Internet plus management platform according to claim 8, wherein the offline chronic disease service center comprises a wearable device experience center and a contract consulting desk, and the online home health service system comprises a personal file module, a member incentive module, an online mall, a video consultation module, a data monitoring module and a health education module.

13. An operating method of the chronic pain Internet plus management platform according to claim 1, comprising:
S101: establishing resident health files, and intelligently screening chronic diseases of target population, comprising
establishing an Internet of Things collection system, based on the resident health files and a screening standard for chronic pain high-risk population, to improve resident health information and provide data support for hierarchical diagnosis and treatment; collecting questionnaire data corresponding to high-risk conditions and a set of sample data about whether a sample is diagnosed as the chronic pain in a sample population, and combining the questionnaire data and the set of sample data with the resident health files to form the sample data;
S102: sharing information between an Internet analysis and diagnosis platform and a region medical information system, comprising
establishing the Internet hierarchical diagnosis and treatment platform and interconnecting the resident health files and electronic cases with the Internet hierarchical diagnosis and treatment platform to realize medical information sharing and medical service collaboration in a region; and interconnecting the hierarchical diagnosis and treatment platform with existing region health information platforms, all-level medical institutions and region medical central business systems to assist doctors to easily access to anamnesis and specific conditions about diagnosis and treatment of patients through platforms, and provide data support for implementation of the hierarchical diagnosis and treatment; and
establishing a standard knowledge system and service process and an uniform diagnosis and treatment standard and operating specification to improve service quality of primary medical institutions; transforming an original one-to-one service mode for large medical institutions and primary medical institutions to a many-to-many service mode by support of a new generation of information technology, so as to enable the large medical institutions to focus on solving intractable diseases and critical illnesses, enable the primary medical institutions to emphatically focus on common diseases, chronic diseases, frequently-occurring diseases and rehabilitation services, to support construction of a hierarchical diagnosis and treatment mode for separate treatment of acute and chronic diseases, primary diagnosis, dual referral and superior-subordinate collaboration;
S103: assisting diagnosis and treatment of chronic pain by interoperability of platforms, comprising
assisting patients to be diagnosed by platform, comprising: based on construction of a patient information platform and an Internet triage platform, realizing collaboration medical services for intelligent triage, appointment for registration, appointment for diagnosis and treatment, remote outpatient service, telemedicine, dual referral/inspection, remote instruction, and communication between doctors and patients, and between doctors and doctors; and assisting doctors in task management, task recommendation and automatic distribution, by an informatization platform and a method for intelligent recommendation and standardized process design, to improve standardized diagnosis and treatment capabilities of primary doctors for the chronic pain; and
assisting primary doctors in precise diagnosis and treatment, comprising: based on an assessment for conditions of a patient, assessing a disease group that the patient belongs to, comprehensively assessing a disease level and a treatment plan by combining with information of a platform database, and formulating a medication recommendation based on recommendations given by the platform and a hospital situation, and submitting data; establishing files of respective primary patients, comprising information about a health file, an electronic case, clinical features, the disease group, the medication recommendation, a condition whether the disease is acutely exacerbated and a condition whether there is a complication, to form a systematic diagnosis and treatment report, and building an acute exacerbation risk model and a complication occurrence risk model based on patient information and different medication plans to formulate a medication plan; modeling abnormal data, based on data of the resident health files, medication situation and medication effect for patients accumulated in each region, in combination with abnormal data beyond medication specification, and building a hybrid model supporting grouping of intractable diseases and an abnormal modeling model, to precisely recommend medication; and
S104: performing intelligent medication guidance and auxiliary follow-up for chronic pain, comprising
formulating an optimum medication plan by cloud computing, based on follow-up information comprising the resident health files, the clinical features, the medication situation and a condition whether the disease is acutely exacerbated, in combination with information of the database to give patients medication guidance at any time, and performing regular auxiliary follow-up according to cycles of different chronic pain diseases, and recording and uploading patient information to the cloud to formulate an appropriate recommendation for follow-up plan.

14. The operating method of the chronic pain Internet plus management platform according to claim 13, further comprising: a chronic disease database, a region chronic disease support platform, a hospital chronic disease business subsystem, a community hospital chronic disease business subsystem, an offline chronic disease service center, and an online home health service system, wherein
the chronic disease database exchanges data with the region chronic disease support platform mutually;
the hospital chronic disease business subsystem, the community hospital chronic disease business subsystem and the online home health service system exchange data respectively with the region chronic disease support platform mutually;
the hospital chronic disease business subsystem is controlled and managed by a region central hospital;
the community hospital chronic disease business subsystem is controlled and managed by a community health service center; and
the offline chronic disease service center and the online home health service system provide service for home patients.

15. The operating method of the chronic pain Internet plus management platform according to claim 14, wherein the region chronic disease support platform comprises a telemedicine module, a remote electrocardiogram module, a remote image module, a decision and analysis module, an artificial intelligence AI engine, a dual referral module and a data integration platform.

16. The operating method of the chronic pain Internet plus management platform according to claim 14, wherein the hospital chronic disease business subsystem comprises an online prescription module, an online diagnosis and treatment module, a treatment plan formulation module and a specialist follow-up module.

17. The operating method of the chronic pain Internet plus management platform according to claim 14, wherein the community hospital chronic disease business subsystem comprises an electronic filing module, a treatment plan execution module, an outpatient counterparty module and a chronic disease management module.

18. The operating method of the chronic pain Internet plus management platform according to claim 14, wherein the offline chronic disease service center comprises a wearable device experience center and a contract consulting desk, and the online home health service system comprises a personal file module, a member incentive module, an online mall, a video consultation module, a data monitoring module and a health education module.
